(19) United States
(12) Patent Application Publication
(10) Pub. No.: US 2005/0233915 A1
(43) Pub. Date: Oct. 20, 2005

(54) Foaming soap, and methods

(75) Inventor: Kim R. Smith, Woodbury, MN (US)

Correspondence Address: ECOLAB INC., MAIL STOP ESC-F7, 655 LONE OAK DRIVE, EAGAN, MN, 55121, US

(73) Assignee: Ecolab Inc.

(21) Appl. No.: 10/825,389

(22) Filed: Apr. 15, 2004

(51) Int. Cl. A61K 7/50

(52) U.S. Cl. 510/130

(57) ABSTRACT

A liquid soap, as defined by 21 C.F.R. 701.20, that is contained in a dispensing container having a foam dispenser head. When dispensed, the foam dispenser head combines the soap with air to provide a foam. The soap is free of any additive, such as moisturizer, thickener, or the like, which would remove the soap from the FDA “soap” definition. The soap is dilutable with a solvent, such as water.

### FIELD

The present invention relates to soap, particularly, to dispensable, foaming soap.

### BACKGROUND

With the growing awareness regarding the spread of diseases in today's society, cleanliness has gained high importance. In many occupations, washing of the hands is mandated either by state or federal law; for example, employees in the restaurant business are required to wash their hands with soap and water after using the rest room; food service employees are required to wash their hands after handling money. Even if not required, many people are concerned about the cleanliness of their hands after doing common activities, such as opening doors, holding handrails, and even touching items that have been touched by other people. Frequent washing of one's hands, along with other body parts, is common.

To facilitate this frequent washing, many detergents and skin cleansing products are provided in a liquid form, dispensed by a pump dispenser.

It is a consumer conception that washing hands with a cleanser having foaming or lathering ability increases the resulting cleanliness of the hands. This conception may be based on the consumer's recollection of the lather produced by using a cleanser in conjunction with a bristled scrubbing brush, or, that the lather easily contacts all surfaces of the hands, thus better cleansing them. In an attempt to provide the sense of cleanliness while maintaining a sense of luxury, liquid cleanser product have been created that provide a rich, foamy lather upon dispensing. The rich, foamy lather is generally a combination of the cleanser composition and the disperser used to dispense the liquid composition.

Many liquid cleansing compositions, such as handsoap or other skin cleaners, although commonly referred to as “soap”, do not meet the official definition of “soap”. “Soap”, as defined by 21 Code of Federal Regulations 701.20, has three basic requirements: the bulk of the non-volatile matter must consist of alkali salts of free fatty acids; the detergent properties must be due to the alkali-fatty acid compounds; and the product must be labeled, sold, and represented as “soap”. Excluded is the incorporation of a deliberately added ingredient for the moisturization, sanitation, or health of the skin. A common “soap” is Proctor & Gambles IVORY bar soap.

“Soap” does not equal “handsoap”, “skin cleanser”, and the like. There are many handsoaps and skin cleaners that do not meet the requirements for being called “soap”. These non-qualifying cleansers, although intended to be used as soap to cleanse body parts, are regulated by the Food and Drug Administration as a “cosmetic”. Many of these cosmetic grade so-called soaps have foam boosters to facilitate dispensing the material as a foam through foamer heads.

What is desired is a liquid cleansing product that is dispensable through a foamer head and has foaming and lathering properties, but that does not fall within the definition of a cosmetic and thus regulation by the FDA; that is, the foamable product is “soap”.

### SUMMARY OF THE DISCLOSURE

The present invention relates to dispensable liquid soap products that are foamable, and methods of dispensing the diluted soap as a foam. Also included are methods of diluting a concentrated soap composition to form a diluted soap composition that is dispensable and foamable.

The soap composition is one that is defined as a “soap” by 21 C.F.R. 701.20, having the bulk of the non-volatile matter being alkali salts of free fatty acids and the detergent properties of the soap due to those alkali salts, and being free of additives for the moisturization, sanitation, or health of the skin. The soap may be ready-to-dispense or may be present as a concentrate that is diluted at either the point-of-use or point-of-sale.

The invention is directed to a product comprising a soap, as defined by 21 C.F.R. 701.20, present in a liquid container including a foam dispenser head, which produces a foam or lather by mixing the soap with air. The liquid container can be a stand-alone, disposable product or be a refillable, permanent product.

In another particular embodiment, the invention is directed to a method of using a soap product, the method including providing a soap as defined by 21 C.F.R. 701.20, and dispensing the soap from a dispenser with air to create a foam. In another particular embodiment, the method includes providing a concentrated soap as defined by 21 C.F.R. 701.20, diluting the soap with water to form a diluted soap, and dispensing the diluted soap from a dispenser with air to create a foam.

Additional details regarding the methods and the compositions are provided below.

### BRIEF DESCRIPTION OF THE FIGURE

FIG. 1 is perspective view of one embodiment of a liquid soap dispenser having soap therein.

### DETAILED DESCRIPTION OF THE INVENTION

Soap, as defined by the United States Code of Federal Regulations, 21 C.F.R. 701.20, has three basic requirements: the bulk of the non-volatile matter must consist of alkali salts of free fatty acids; the detergent properties must be due to the alkali-fatty acid compounds; and the product must be labeled, sold, and represented as “soap”. Excluded is the incorporation of a deliberately added ingredient for the moisturization, sanitation, or health of the skin. Ingredients commonly found in many skin cleansers and handsoaps, which are not allowed in “soap”, include thickeners such as methyl cellulose, petroleum products, aloe, and the like. These ingredients are present in the cleansers to provide a more aesthetically pleasing (e.g., due to dyes or fragrances), more luxurious (e.g., due to foaming enhancers), cleanser. Many of today's consumer detergents and skin cleaning products do not qualify as “soap”.

Skin cleaners which do not qualify as “soap” are regulated by the Food and Drug Administration (FDA) as “cosmetics” under the Food, Drug, and Cosmetic Act of 1938. The term “cosmetic” means (1) articles intended to be rubbed, poured, sprinkled, or sprayed on, introduced into, or otherwise applied to the human body or any part thereof for cleansing, beautifying, promoting attractiveness, or altering the appearance, and (2) articles intended for use as a component of any such articles; except that such term shall not include soap. This Act is further detailed in 21 C.F.R. 700-740. “Soap”, of 21 C.F.R. 701.20, is specifically excluded from the definition of “cosmetic” and is not regulated by the FDA. Non-regulation by the FDA may have benefits for developers, manufacturers, marketers, and sellers of products.

To increase the consumer's conception of a rich, luxurious and/or moisturizing cleanser, many handsoaps and other detergents are deliberately thickened. These thickened cleansers have a viscosity of greater than 100 cps, usually greater than 150 cps. Such thickened soaps cannot be dispensed through a foamer head due to their high viscosities. A few low viscosity liquid handsoaps, such as DIAL COMPLETE, a medicated handsoap, or DIGICLEAN SLIMLINE from Ecolab Inc., a cosmetic handsoap, have a viscosity less than 100 cps and are dispensed as foams. However, these thin handsoaps do not meet the FDA's definition of “soap” and are subject to cosmetic regulations.

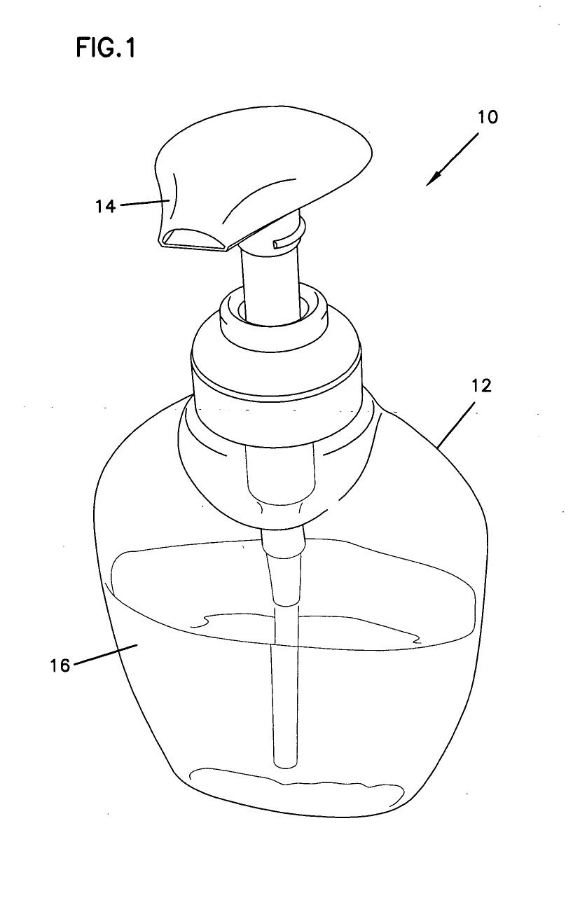

A trend for consumer skin cleansers has been to package the liquid cleanser in a pumpable liquid dispenser. One embodiment of such a dispenser is shown in FIG. 1, described below. Such dispensers, and alternate embodiments of the dispenser shown, are well known. Manufacturers have found that, in general, the pumpable liquid dispensers work well with thick cleansers (having viscosity greater than 100 cps).

In an attempt to further increase the conception of a rich and luxurious cleanser, some thinner or lower viscosity skin cleanser products have been packaged in dispensers that include a foam dispenser head that creates foam or lather without the use of gas propellants. Rather than using a pressurized gas to create the foam, readily available air is mixed with the liquid being dispensed, by the dispenser head, to provide the foam. The skin cleansers generally include additives to provide a consistency that will produce the desired foam. Additives, such as synthetic anionic and, often amphoteric, surfactants are added to enhance the foam. Examples of commercially available skin cleanser products using the foam dispenser heads include SOFTSOAP Foam Works from Colgate-Palmolive, and products from Dial and from Johnson & Johnson. These products are not “soap”, but are “cosmetics” regulated by the FDA.

What is desired is a non-cosmetic product which can be dispensed through a foamer head and which does not require “cosmetic” testing such as multi-year long shelf life tests, detailed health and safety testing (such as animal testing), and specific labeling requirements. The soap product of the present invention, a foamable, dispensable “soap”, packaged in a liquid container having a foam dispenser head, meets these desires.

Referring to FIG. 1, a packaged soap product 10 is illustrated. Product 10 includes a liquid container 12 and a foam disperser head or foamer head 14. A volume of soap 16 is present in container 12. While within container 12, soap 16 is a liquid. Upon dispensing through foamer head 14, the expelled soap is a foam.

Soap 16, a “soap”, meets the requirements of 21 C.F.R. 701.20, and has the bulk of the non-volatile matter being alkali salts of free fatty acids, the detergent properties being due to the alkali-fatty acid compounds. The product 10, including soap 16, is labeled, sold, and represented as “soap”.

At least 50 wt-% of the nonvolatile solids in soap composition are alkali salts of fatty carboxylic acids. Preferred alkali salts include sodium, potassium and magnesium, either individually or as mixtures thereof. Potassium salts are particularly preferred. The fatty acids may be C6 through C24, or blends thereof, and may optionally be fully or partially hydrogenated. In some embodiments, at least 75 wt-% of the nonvolatile solids are alkali salts of fatty carboxylic acids. Preferably, this level is al least 85 wt-%. The detergent properties of the soap are due to these fatty acids.

The soap does not include any synthetic surfactants such as sulfates, sulfonates, isothioates, amine oxides, betaines, and the like.

The soap may include glycerin, which is a by-product generated during saponification of fatty glyceride to alkali carboxylate. No additional glycerin or other ingredient deliberately added for the moisturization or health of the skin is present in the soap.

The incorporation of additional ingredients, which are consistent with the requirement for chemical identity and use levels defined in 21 C.F.R. 701.20, may be present in the soap. Examples of allowed ingredients include antioxidants (such as vitamin C), water softening agents (such as EDTA), preservatives (such as paraben), solubilizers (such as propylene glycol), colors (pigments or dyes), fragrances, pH modifiers (such as citric acid), and solvents (such as water, ethanol, propanol, isopropyl alcohol, and butanol).

Examples of “soaps” that are suitable for use in the packaged product include “LiquiSan C” from Ecolab, Inc., “Norfox Coco Power”, “Norfox KO”, “Norfox Oleic Flakes”, “Norfox Oleic Powder”, “Norfox Oleic Powder WSA”, “Norfox 1101” and “Norfox 1115” from Norman, Fox & Company, and potassium cocoate, potassium oleate and sodium laurate from Chemron Corp.

The soap may be present in the packaged product as manufactured (or received from the manufacturer), or, the soap may be diluted to lower the percentage solids. That is, the soap may be obtained ready-to-dispense or may be present as a concentrate that is diluted at either the point-of-use or point-of-sale. Water is the preferred solvent for dilution.

The water can be any suitable form, such as tap water, softened water, unsoftened water, and deionized water. A preferred water for use in the soap product is softened water, typically having a less than 20 grain hardness, more typically less than 15 grain hardness.

A concentrated soap composition, which is intended to be diluted, has at least 10 wt-% solids and usually has at least 20 wt-% solids. In a preferred embodiment, the concentrated soap has at least 35 wt-% solids. Preferably, concentrated soap is diluted at least 1:1 soap:water, more preferably at least 1:4, and most preferably at least 1:6. With some concentrated soaps, such as a 40% solution of potassium cocoate, a dilution of 1:5-1:10 is preferred.

After dilution, the soap, as packaged, will have at least 1 wt-% solids and generally no more than 20 wt-% solids. A preferred range for the amount solids in the packaged liquid soap is about 2 wt-% to 8 wt-%. The soap, as packaged, will generally have a viscosity less than 100 cps, often less than 90 cps.

The soap 16, optionally diluted with water, is contained in a dispenser having liquid retaining container 12 and foam dispenser head 14, as illustrated in FIG. 1. A tube, pipette, straw, or other such means is present to fluidly connect the soap 16 to the dispenser head 14. The product 10 can be a stand-alone, disposable product (as illustrated in FIG. 1) or be a refillable, permanent dispenser, such as mounted on a wall. The liquid retaining container 12 can be any suitable package onto which the foam dispenser head 14 can be attached, either removably or permanently.

The foam dispenser head 14 combines the soap 16 in the container 12 with air. In order to provide the desired foam consistency, the foam dispenser head 14 generally has an air chamber with a liquid chamber running through it. When the dispenser head is activated, typically pumped, both air and soap are dispensed simultaneously to create the foam. The dispenser head design allows for the precise mixing of soap and air, resulting in a dose of high quality foam with each single stroke.

Various types of foam dispenser heads that create foam without the use of gas propellants are available from Airspray N.V., of the Netherlands, and their U.S. subsidiary Airspray International of Pompano Beach, Fla. Examples of various designs of foaming heads are disclosed in U.S. Pat. Nos. 6,536,629; 6,053,364; 5,429,279; 5,337,929; Des. 452,822; and Des. 452,653.

The present invention can be better understood with reference to the following examples. These examples are intended to be representative of specific embodiments of the invention, and are not intended as limiting the scope of the invention.

### EXAMPLE 1

LiquiSan C, a 40% active solution of potassium cocoate (available from Ecolab, Inc. of St. Paul, Minn.) was diluted to 4% solids with tap water. The solution was dispensed through a foam dispenser head (available from Airspray International).

The resulting foam was shaving cream-like quality, but met the chemical requirement for FDA classification as “soap”.

### EXAMPLE 2

A liquid composition (16 wt-% LiquiSan C, 0.1 wt-% methyl paraben, 0.7 wt-% isopropyl alcohol, and 83.2 wt-% water) was prepared. The liquid had a viscosity of less than 100 cps. The solution was dispensed through a foam dispenser head (available from Airspray International).

The resulting foam was shaving cream-like quality, but met the chemical requirement for FDA classification as “soap”.

It should be noted that, as used in this specification and the appended claims, the singular forms “a,” “an” and “the” include plural referents unless the content clearly dictates otherwise. It should also be noted that the term “or” is generally employed in its sense including “and/or” unless the content clearly dictates otherwise.

The invention has been described with reference to various specific and preferred embodiments and techniques. However, it should be understood that many variations and modifications may be made while remaining within the spirit and scope of the invention.

1. A soap product comprising:
(a) a dispenser comprising a liquid retaining container and a foam dispenser head; and
(b) liquid soap present in the container, the foam dispenser head fluidly connected to the liquid soap.

2. The soap product according to claim 1, wherein the soap comprises non-volatile matter consisting essentially of alkali salts of free fatty acids.

3. The soap product according to claim 2, wherein the alkali salts are selected from the group consisting of sodium, potassium, magnesium, and mixtures thereof.

4. The soap product according to claim 2, wherein the soap comprises a mixture of concentrated soap and water.

5. The soap product according to claim 4, wherein the mixture of concentrated soap and water comprises 1:1 to 1:10 concentrated soap:water.

6. The soap product according to claim 4, wherein the concentrated soap comprises at least 50 wt-% alkali salts.

7. The soap product according to claim 6, wherein the concentrated soap comprises at least 75 wt-% alkali salts.

8. The soap product according to claim 4, wherein the concentrated soap comprises at least 10 wt-% solids.

9. The soap product according to claim 8, wherein the concentrated soap comprises at least 20 wt-% solids.

10. The soap product according to claim 2, the soap further comprising at least one of an antioxidants, a water softening agents, a preservative, a solubilizers, a dye, a fragrance, or a pH modifier.

11. A method of dispensing a soap product, the method comprising:
(a) providing a concentrated soap;
(b) diluting the soap with water to form a diluted soap; and
(c) dispensing the diluted soap from a dispenser with air to create a foam.

12. The method according to claim 11, wherein the step of diluting the soap with water to form a diluted soap is done before the step of dispensing the diluted soap from a dispenser with air to create a foam.

13. The method according to claim 11, wherein the step of dispensing the diluted soap from a dispenser with air to create a foam comprises:
(a) mixing the soap with air in a foam dispenser head to create a foam; and
(b) dispensing the foam via the foam dispenser head.

14. A method of dispensing a soap product, the method comprising:
(a) providing a soap; and
(b) dispensing the soap from a dispenser with air to create a foam.

15. The method according to claim 14, wherein the step of dispensing the soap from a dispenser with air to create a foam comprises:
(a) mixing the soap with air in a foam dispenser head to create a foam; and
(b) dispensing the foam via the foam dispenser head.
